(19) United States
(12) Patent Application Publication
(10) Pub. No.: US 2014/0194803 A1
(43) Pub. Date: Jul. 10, 2014

(54) WOUND-HEALING COMPOSITIONS AND METHOD OF USE

(71) Applicants: Patrick J. Parks, Mendota Heights, MN (US); Marnie L. Peterson, Golden Valley, MN (US); Michele J. Anderson, Minneapolis, MN (US)

(72) Inventors: Patrick J. Parks, Mendota Heights, MN (US); Marnie L. Peterson, Golden Valley, MN (US); Michele J. Anderson, Minneapolis, MN (US)

(73) Assignees: REGENTS OF THE UNIVERSITY OF MINNESTOA, MINNEAPOLIS, MN (US); 3M INNOVATIVE PROPERTIES COMPANY, ST. PAUL, MN (US)

(21) Appl. No.: 14/235,194

(22) PCT Filed: Jul. 23, 2012
(86) PCT No.: PCT/US2012/047786

§ 371 Date: Jan. 27, 2014

Related U.S. Application Data
(60) Provisional application No. 61/512,655, filed on Jul. 28, 2011.

(51) Int. Cl. A61L 26/00; A61K 31/155; A61K 33/42

(52) U.S. Cl. CPC A61L 26/0066; A61K 33/42; A61K 31/155
USPC 602/48; 424/601

(57) ABSTRACT

A composition and wound dressing for treating a skin wound is disclosed. The composition and dressing comprise a pharmaceutically acceptable carrier; an effective amount of an active ingredient of inorganic solids comprising a potassium salt, a zinc salt, a calcium salt, and a rubidium salt; and an antimicrobial biguanide compound comprising an amount effective to reduce the number of viable microorganisms at a wound site. Methods of use include contacting a wound site with the composition and/or dressing.

### CROSS REFERENCE TO RELATED APPLICATIONS

This application claims the benefit of U.S. Provisional Patent Application No. 61/512,655, filed Jul. 28, 2011, which is incorporated herein by reference in its entirety.

### BACKGROUND

Compositions of aqueous oak bark extract, and synthetic compositions containing the key active ingredients of oak bark extract, have been disclosed for the treatment of a variety of skin conditions, including fungal infections, minor infections, insect bites, minor burns, sunburn, poison oak, poison ivy, poison sumac, wound healing, pyodermas, dermatitis, pruritic dermatoses, eczema, decubitus ulcers, tropical ulcers, decubitus, psoriasis, impetigo, Kaposi sarcoma, warts, gangrene, ischemic ulcer, keratosis, and skin cancer.

Oak bark extract has been described in U.S. Pat. No. 5,080,900 which is incorporated herein by reference, for use in the treatment of skin ulcers, particularly decubitus ulcers or bed sores. This material in a base of WHITFIELD pharmaceutical ointment has also been sold under the trade name BENCELOK® for use in the treatment of minor skin irritations (Whitfield and Bencelok are trademarks for pharmaceutical ointments). The amount of oak bark extract in these materials was relatively low, however. For example, the BENCELOK® preparations have contained from 0.25 to 3% by weight of ash-derived components based upon the total weight of the preparation.

Certain skin wounds fail to maintain a progression of biological events that ultimately lead to healing of the wounds. These skin wounds are designated as “stalled wounds”. The fundamental cause(s) of this lack of healing is not well-understood. There exists a need for a treatment for stalled wounds.

### SUMMARY

In general, the invention generally relates to the treatment of skin wounds (e.g., surgical wounds, pressure ulcers, incisions, abrasions, and the like). In particular, the present disclosure relates to compositions and dressings and methods of use thereof to treat a skin wound. The inventive compositions comprise an antimicrobial biguanide compound and an active ingredient of inorganic solids comprising salts of potassium, zinc, calcium, and rubidium. The inventive compositions, which can be used alone or in combination with a wound dressing, directly and/or indirectly facilitate biological processes associated with wound healing.

In one aspect, the present disclosure provides a composition. The composition can comprise a pharmaceutically acceptable carrier, an effective amount of an active ingredient of inorganic solids, and a biguanide compound comprising an amount effective to reduce the number of viable microorganisms at a wound site. The active ingredient can comprise a potassium salt, a zinc salt, a calcium salt, and a rubidium salt, wherein each of the salts comprises a pharmaceutically-acceptable anion.

In any embodiment of the composition, the active ingredient of inorganic solids can comprise 10-80 parts of potassium ions, 0.00001-20 parts of zinc ions, 0.01-10 parts of calcium ions and rubidium ions in an amount up to 40 parts, said parts being expressed as parts by weight of inorganic solids. In any of the above embodiments, the biguanide compound can be selected from the group consisting of polyhexamethylene biguanide, chlorhexidine, octenidine, derivatives thereof, and combinations thereof. In any of the above embodiments, relative to a composition without the biguanide component, the composition with the biguanide compound can suppress the accumulation of a polypeptide associated with inflammation. In some embodiments, relative to a composition without the active ingredient, the composition with the active ingredient suppresses the accumulation of a polypeptide associated with inflammation.

In another aspect, the present disclosure provides a wound dressing. The wound dressing can comprise any of the above embodiments of the composition and a support. In any embodiment of the dressing, the support can comprise a fibrous material, a film, a gel, a foam, a hydrocolloid, an alginate, a hydrogel a polysaccharide paste, a plurality of granules, a plurality of beads or a combination of any two or more of the foregoing.

In yet another aspect, the present disclosure provides a method of treating a wound. The method can comprise contacting a wound with any of the above embodiments of the composition.

In yet another aspect, the present disclosure provides a method of treating a wound. The method can comprise contacting a wound with any of the above embodiments of the wound dressing.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

A “skin wound”, as used herein, refers to a break in the continuity of the skin barrier that may result from trauma (e.g., a puncture, a laceration, or an abrasion) or surgery, for example.

A “chronic wound”, non-healing wound”, “slow to heal wound”, or “stalled wound”, as used herein, refers to a category of wound that fails to heal over a typical (e.g., 8-12 weeks) timeframe from inception of the wound to complete closure of the skin at the wound site.

The terms “comprises” and variations thereof do not have a limiting meaning where these terms appear in the description and claims.

As used herein, “a,” “an,” “the,” “at least one,” and “one or more” are used interchangeably. Thus, for example, a polypeptide can be interpreted to mean “one or more” polypeptides.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### DETAILED DESCRIPTION

Skin wounds designated as “chronic” or “slow to heal” wounds, “non-healing” wounds or “stalled wounds” and are commonly observed in a clinical setting as venous ulcers, diabetic ulcers, particularly diabetic foot ulcers, or combinations of these two entities. Similar non-healing wounds can be observed in less frequent conditions, e.g., autoimmune disorders but irrespective of the etiology the wounds share the characteristic of failing to heal in a clinically expected timeframe. Empirically, wounds that take longer than 12 weeks to heal from the first observation of a wound are considered to fall into the “chronic” “non-healing”, or “slow to heal” categories. The term “stalled wound” also includes some wounds that fail to heal. However, a “stalled wound” is more commonly associated with a wound that begins to heal but, for unknown reasons, stops healing before complete closure of the wound. The fundamental cause(s) of this lack of healing is not well-understood. There exists a need for a treatment for any of these types of wounds since spontaneous healing has failed to occur.

Excess inflammation, which can be concomitant with microbiological infection, can inhibit normal healing processes in a skin wound. The present disclosure relates to compositions, articles, and methods for treating a skin wound. The compositions and articles include an antimicrobial biguanide compound and an active ingredient of inorganic solids comprising salts of potassium, zinc, calcium, and rubidium. The components of the inventive composition surprisingly provide a synergistic effect that results in a suppression of the accumulation of a biochemical marker (e.g., a polypeptide, a cytokine such as interleukin-8(IL-8), for example) associated with inflammation

Compositions of the present disclosure comprise an active ingredient of inorganic solids. Exemplary active ingredients of inorganic solids of the present disclosure can be found in oak bark ash and related synthetic compositions, as described in U.S. Pat. Nos. 6,149,947 and 7,014,870, which each are incorporated herein by reference in their entirety.

The therapeutic activity of various constituents of oak bark extract has been analyzed and the results indicate therapeutic efficacy for compositions containing just potassium, zinc and calcium ions, in combination with suitable counterions. Thus, synthetic formulations containing, by weight of inorganic solids, 10 to 80 parts potassium ions, preferably 30 to 50 parts; 0.00001 to 20 parts zinc ions, preferably 1 to 10 parts; 0.01 to 10 parts calcium ions, preferably 1 to 5 parts; and 0 to 40 parts rubidium ions, preferably 1 to 30 parts; together with pharmaceutically acceptable counterions (e.g., C⁻, SO₄⁻, CO₃⁻, OH⁻, Br⁻). Optionally, the composition further may comprise up to 5 parts sulfur, in the form of elemental sulfur or sulfate anions. The solution may also contain other inorganic cations, for example, up to 10 parts by weight of inorganic solids of cobalt, copper, iron, manganese, nickel, strontium or aluminum ions; preferably any of the foregoing can be present in the composition up to 1 part by weight. Further, the composition may include a pharmaceutically acceptable carrier such as water or an ointment or cream base which will result in a therapeutic composition having a pH in the range of about 4 to 7, inclusive. Preferably, the composition has a pH in the range of about 4.5 to 5.5, inclusive. As used herein, a composition comprising “PHI salts” refers to a composition that includes potassium, zinc, calcium, and rubidium cations together with pharmaceutically-acceptable counterions.

Oak bark extract or the related synthetic mixtures of inorganic solids have been found to provide a variety of beneficial therapeutic properties for the treatment of a variety of skin conditions, as described herein.

Compositions according to the invention are also useful for treating abrasions and other partial thickness wounds. Useful compositions include at least potassium, zinc and calcium ions, for example. The composition is advantageously applied in a cream or ointment base for a period of time (e.g., several hours to several days).

While not intending to be bound by any particular mechanism of action, it appears that oak bark extract and synthetic mixtures containing the key ingredients of oak bark extract function to enhance wound healing by providing complexing ions which interact with biological membranes and/or enzymes including, but not restricted to, alkaline phosphatase, carbonic anhydrase, carboxypeptidase, various enhydrogenases, arginase, carnosinase, dehydropeptidase, glycine dipeptidase, histidine deaminase and tripeptidase, oxyloacetic carboxylase, and some lecithinases and enolases. These enzymes are involved in numerous biosynthetic pathways necessary for wound healing, for example, collagen biosynthesis, and are believed to function with greater efficiency in the presence of the complexing ions.

Compositions of the present disclosure further comprise an antimicrobial biguanide compound. An exemplary antimicrobial biguanide compound is polyhexamethylene biguanide (PHMB), for example. The use PHMB in a biosynthesized cellulose wound dressing is described by Mulder et al. (“Polyhexamethylene Biguanide (PHMB): Antimicrobial Agents in Wound Care”; Wounds; 19(7):173-182; 2007; which is incorporated herein by reference in its entirety). Other suitable biguanides include, for example, chlorhexidine and octenidine. Compositions comprising combinations of biguanide compounds are also included.

The biguanide compound is present in the composition in an amount effective to provide antimicrobial activity (e.g., bactericidal and/or bacteristatic activity) in the wound site. Compositions of the present disclosure can comprise about 0.1 weight percent to about 0.5 weight percent biguanide compound (e.g. polyhexamethylene biguanide).

One or more of the components of the composition can be dissolved and/or suspended in a pharmaceutically-accepted carrier. In any embodiment, the carrier may be water, a gel, or an ointment (e.g., WHITFIELD'S ointment). “Pharmaceutically-acceptable”, as used herein refers to a carrier that does not include an ingredient that substantially interferes with the healing process of a wound with which the carrier is contacted. In some embodiments, the carrier may also comprise an aqueous solution or suspension of ethylhexylglycerin, cocamidopropyl hydroxysultaine, lactic acid, sodium hydroxide, glycerin, xylitol, polyvinyl pyrrolidone, polyvinyl pyrrolidone, polyethylene glycol 660 hydroxystearate, or a combination of any two or more of the foregoing. Advantageously, the foregoing pharmaceutically-acceptable carriers can also facilitate the penetration of the compositions into the wound and/or provide local pH changes that may benefit the wound healing.

The present disclosure further provides a wound dressing. The wound dressing comprises a support and any of the compositions disclosed herein wherein the composition includes a pharmaceutically-acceptable carrier; an effective amount of an active ingredient of inorganic solids comprising a potassium salt, a zinc salt, a calcium salt, and a rubidium salt; and a biguanide compound comprising an amount effective to reduce the number of viable microorganisms at a wound site. In some embodiments, the composition can be applied to the dressing as a coating (e.g., a coating on a surface of the dressing that, in use, is oriented toward a wound). In some embodiments, the wound dressing may be imbued (e.g., saturated) with the composition.

Wound dressings of the present disclosure may comprise a variety of support materials including, but not limited to, a fibrous material, a film, a gel, a foam, a hydrocolloid, an alginate, a hydrogel a polysaccharide paste, a plurality of granules, a plurality of beads or a combination of any two or more of the foregoing. In some embodiments, the dressing may further comprise a backing (e.g., an adhesive backing to protect the wound dressing and, optionally, provide adhesive secural of the dressing to a patient's skin).

The present disclosure further provides a method of treating a wound. In some embodiments, the method comprises contacting a wound with any composition of the present disclosure wherein the composition includes a pharmaceutically-acceptable carrier; an effective amount of an active ingredient of inorganic solids comprising a potassium salt, a zinc salt, a calcium salt, and a rubidium salt; and a biguanide compound comprising an amount effective to reduce the number of viable microorganisms at a wound site. The composition can be applied to the wound, for example, in a liquid (e.g., by lavaging the wound with the liquid) or in a gel or an ointment. Liquid compositions can provide substantially immediate delivery of the ions directly to the healing tissue. In contrast, gels and ointments can provide delivery of the ions over a sustained period of time. In some embodiments, the composition can be applied to a wound dressing, which is subsequently contacted with the wound. Advantageously, a dressing comprising the composition can be contacted with the wound for a period of time (e.g., several hours to a day, or longer), thereby providing a moist environment enriched with the PHI cations and the antimicrobial biguanide to facilitate healing of the skin.

### Embodiments

Embodiment 1 is a composition, comprising:
a pharmaceutically acceptable carrier;
an effective amount of an active ingredient of inorganic solids comprising a potassium salt, a zinc salt, a calcium salt, and a rubidium salt; and
an antimicrobial biguanide compound comprising an amount effective to reduce the number of viable microorganisms at a wound site;
wherein each of the salts comprises a pharmaceutically-acceptable anion.

Embodiment 2 is the composition of embodiment 1, wherein the active ingredient of inorganic solids comprises 10-80 parts of potassium ions, 0.00001-20 parts of zinc ions, 0.01-10 parts of calcium ions and rubidium ions in an amount up to 40 parts, said parts being expressed as parts by weight of inorganic solids.

Embodiment 3 is the composition of embodiment 1 or embodiment 2, wherein the biguanide compound is selected from the group consisting of polyhexamethylene biguanide, chlorhexidine, octenidine, derivatives thereof, and combinations thereof.

Embodiment 4 is the composition of any one of the preceding embodiments, wherein the composition comprises about 0.1 weight percent to about 0.5 weight percent of the biguanide compound.

Embodiment 5 is the composition of any one of the preceding embodiments wherein, relative to a composition without the biguanide component, the composition with the biguanide compound suppresses the accumulation of a polypeptide associated with inflammation.

Embodiment 6 is the composition of any one of embodiments 1 through 4 wherein, relative to a composition without the active ingredient, the composition with the active ingredient suppresses the accumulation of a polypeptide associated with inflammation.

Embodiment 7 is the method of embodiment 5 or embodiment 6, wherein the polypeptide associated with inflammation comprises a cytokine.

Embodiment 8 is the method of embodiment 7, wherein the cytokine comprises interleukin-8.

Embodiment 9 is a wound dressing, comprising:
the composition of any one of the preceding embodiments; and a support.

Embodiment 10 is the wound dressing of embodiment 9, wherein the support comprises a fibrous material, a film, a gel, a foam, a hydrocolloid, an alginate, a hydrogel a polysaccharide paste, a plurality of granules, a plurality of beads or a combination of any two or more of the foregoing.

Embodiment 11 is the wound dressing of embodiment 9 or embodiment 10, further comprising a backing layer.

Embodiment 12 is a method of treating a wound, comprising contacting a wound with the composition of any one of embodiments 1 through 8.

Embodiment 13 is a method of treating a wound, comprising contacting a wound with the wound dressing of any one of embodiments 9 through 11.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

Materials. Materials utilized in the preparation of the examples are shown in Tables 1-3.

TABLE 1
Materials Table

| Component | Description | Source |
|---|---|---|
| Normal porcine vaginal | Full-thickness | University of |
| mucosa | squamous | Minnesota, St. |
| | epithelium | Paul, MN |
| RPMI-1640 | Cell culture media | Invitrogen Life |
| | | Technologies; |
| | | Carlsbad, CA |
| Todd-Hewitt Broth (TH) | Bacteria culture media | Becton Dickinson; |
| | | Franklin Lakes, NJ |
| Phosphate Buffered Saline | Isotonic buffer | Sigma-Aldrich; |
| (PBS) | | St. Louis, MO |

TABLE 2
PHMB Neutralizer Solution Ingredients

| Component | Description | Source | g/L |
|---|---|---|---|
| Potassium | Potassium Phosphate, | JT Baker | 0.4 |
| Phosphate, | Monobasic | | |
| Monobasic | | | |
| Sodium | Sodium Phosphate, Dibasic | Sigma | 10.1 |
| Phosphate, | | | |
| Dibasic | | | |
| Triton X-100 | Polyethylene glycol tert- | Aldrich | 1.0 |
| | octylphenyl ether | | |
| Lecithin | Phosphatidylcholine | Alpha Aesar | 3.0 |
| Tween 80 | Polyethylene glycol sorbitan | Fluka | 30.0 |
| | monooleate | | |
| Sodium | Sodium Thiosulfate, | Sigma- | 1.0 |
| Thiosulfate, | pentahydrate | Aldrich | |
| pentahydrate | | | |
| Water | Water (distilled) | Tap | q.s. to 1 L |

TABLE 3
PHMB Gel Ingredients

| Component | Description | Source | Weight % |
|---|---|---|---|
| Cosmocil CQ | Polyhexamethylene | Arch Chemicals | 2.5 |
| (20% PHMB) | Biguanide (PHMB) | | |
| Sensiva SC 50 | Ethylhexylglycerin | Schulke and Mayr | 0.3 |
| Mackam 50 SB | Cocamidopropyl | Rhodia Novecare | 2.5 |
| | Hydroxysultaine | | |
| HiPure 90 | Lactic Acid | Purac | 5 |
| Sodium | Sodium Hydroxide | Spectrum | 0.8 |
| Hydroxide | | Chemicals | |
| Glycerin | Glycerin | Spectrum | 5 |
| | | Chemicals | |
| Xylasorb | Xylitol | Roquette Pharma | 5 |
| PVP-K90 | Polyvinyl Pyrrolidone | BASF | 2 |
| Tylose H | Hydroxyethylcellulose | ShinEtsu | 1 |
| 4070 NG | | | |
| Solutol HS-15 | Polyethylene Glycol 660 | BASF | 1 |
| | Hydroxystearate | | |
| Water | | | 75 |

### MSSA Preparation

Methicillin sensitive S. aureus (MSSA, strain MN8, a Toxic Shock Syndrome Toxin 1 (TSST-1⁺)-producing clinical mucosal isolate) was streaked onto an agar plate from frozen stock. From this agar plate, an overnight bacterial culture was prepared in TH broth. The culture was washed and diluted in fresh RPMI 1640 to a concentration of about 5×10⁸ CFU/mL.

### Tissue Preparation

Specimens of normal porcine vaginal mucosa were excised from animals at slaughter and rinsed with RPMI-1640. Tissue plugs of uniform size were obtained from the porcine vagina tissue using a 5 mm biopsy punch. Excess muscle tissue was trimmed away with a scalpel. Tissue explants were washed in RPMI-1640 media. The explants were placed, mucosal side up, in a sterile petri dish and washed with fresh RPMI-1640. Explants were incubated at 37 C for 30 minutes, then transferred, mucosal side up, to a 6 well plate with 0.4 μm cell culture transwell inserts containing 0.75 mL RPMI-1640.

A pipette was used to apply the diluted MSSA preparation to the mucosal surface of each tissue sample. After incubation for 2 hours at 37 C, the infected tissues were removed from the incubator and treated with TEGADERM Matrix (3M Health Care, St. Paul, Minn.) and/or PHMB Gel.

### PHMB Gel

PHMB gel was prepared with the ingredients in Table 3. Briefly, all of the ingredients, with the exception of hydroxyethylcellulose and glycerol, were mixed with about 80% of the water. This mixture was stirred until clear, and then the pH was adjusted to 3.5 with lactic acid or sodium hydroxide. The remaining water was added to the mixture. The glycerol and hydroxyethylcellulose were mixed to form a slurry and the slurry was added to the aqueous mixture while stirring. The resulting solution was allowed to stir and thicken overnight at room temperature.

### Example 1

### Treatment of Tissue Explant with a Dressing Comprising an Active Ingredient of Inorganic Ions (PHI Salts) and a Composition Comprising a Biguanide Compound

Porcine vaginal tissue was infected with MRSA as described above. Ten microliters of PHMB gel were then applied to the tissue Immediately following application of the PHMB gel, a small square (0.5 cm²) of TEGADERM Matrix dressing was applied to the PHMB gel on the tissue. Tissue explants were returned to 37 C incubator for 18 h. Two separate experiments were run, each experiment using explant material from a different animal. Samples from the separate experiments were subjected to the Microbial Inhibition Test and the Interleukin-8 Accumulation Test (described below) and the results of the separate experiments are shown in Tables 4 and 5, respectively.

### Comparative Example 1

### Treatment of Tissue Explant with a Dressing Comprising an Active Ingredient of Inorganic Ions

A small square (0.5 cm²) of TEGADERM Matrix dressing was applied to the MSSA-infected tissue. The tissue explants were returned to 37 C incubator for 18 h. Two separate experiments were run, each experiment using explant material from a different animal Samples from the separate experiments were subjected to the Microbial Inhibition Test and the Interleukin-8 Accumulation Test (described below) and the results of the separate experiments are shown in Tables 4 and 5, respectively.

### Comparative Example 2

### Treatment of Tissue Explant with the PHMB Gel

Porcine vaginal tissue was infected with MRSA as described above. Ten microliters of PHMB gel were then applied to the tissue. After application of the PHMB gel, the tissue explants were returned to 37 C incubator for 18 h. Samples were subjected to the Microbial Inhibition Test and the Interleukin-8 Accumulation Test (described below) and the results are shown in Table 5.

### Controls

The control for the microbial inhibition test consisted of agar plates, infected as described above, with no PHI and no PHMB applied.

The control for the IL-8 test consisted of vaginal tissue, infected with MRSA as described above, with no PHI and no PHMB applied. A background reading was also recorded, which consisted of IL-8 concentrations from non-infected vaginal tissue.

### Test Methods

Microbial Inhibition Test

After treatment with TEGADERM Matrix and/or PHMB Gel as described in the Example section and after the incubation step, explants were removed from the transwells and homogenized with 250 μl of PHMB Neutralizer Solution. Serial dilutions of this homogenized tissue were prepared in sterile PBS and plated on Tryptic Soy Agar with 5% sheep blood (Becton Dickinson). After incubating the plates at 37° C. for 24 hours, the number of bacterial colonies on each plate was counted and recorded.

Interleukin-8 (IL-8) Accumulation Test

Samples, prepared as described in the Microbial Inhibition Test, were placed in 250 μl of PBS buffer, homogenized, and centrifuged to clear. The supernatant was transferred to a 1.5 mL tube and frozen until the ELISA analysis. A Porcine IL-8 ELISA Development kit (catalog number DY535; R&D Systems; Minneapolis, Minn.) was utilized to measure IL-8 in the supernatant from homogenized tissue. Note, because the undiluted samples of PHMB in the PHMB neutralizing solution interfered with the ability to detect IL-8, these samples were diluted 1:4 with sterile water before measuring the IL-8.

TABLE 4
Results of the Microbial Inhibition and IL-8 Accumulation tests
for first set of tissue explants. All results shown are based on the
average value measured in duplicate test samples. The number
of micrograms in parentheses refers to the amount of PHMB
gel applied to the tissue explants.

| | Microbial Inhibition* | |
|---|---|---|
| | (log₁₀ reduction | |
| Sample | of CFU/g tissue) | IL-8 Accumulation |
| Control | — | 3907 ± 941 |
| Comparative Example 1 | −2.15 | 693 ± 363 |
| Example 1 (1.2 μg) | 4.33 | 16 ± 3 |
| Example 1 (2 μg) | 1.88 | 0 ± 0 |
| Example 1 (4 μg) | 1.27 | 18 ± 19 |

*Microbial Inhibition is reported as the log₁₀ reduction in the number of MSSA colony-forming units compared to the colony counts on the control plates.

TABLE 5
Results of the Microbial Inhibition and IL-8 Accumulation tests
for second set of tissue explants. With the exception of the background,
all results shown for the microbial inhibition test include the average
and standard deviation of duplicate tests.

| | Microbial Inhibition* | |
|---|---|---|
| | (log₁₀ reduction | |
| Sample | of CFU/g tissue) | IL-8 Accumulation |
| Control | — | 4674 ± 1527 |
| Comparative | −5.26 | 52 ± 201 |
| Example 1 | | |
| Comparative | 0.73 | 3740 ± 1428 |
| Example 2 | | |
| Example 1 | 2.47 | 0** |

*Microbial Inhibition is reported as the log₁₀ reduction in the number of MSSA colony-forming units compared to the colony counts on the control plates.
**The absorbance readings obtained for these samples were less than the background controls and, thus, the IL-98 accumulation is reported as “0”.

The results, shown in Tables 4 and 5, indicate that applying compositions comprising the PHI salts with the PHMB resulted in a 1.27-4.33 log₁₀ reduction in the number of bacteria on the infected tissue, compared to the control that was not treated with PHI salts or PHMB. In contrast, infected tissue that was treated only with the PHI salts did not appear to inhibit the bacteria and the infected tissue that was treated only with the PHMB gel resulted in a smaller log₁₀ reduction (0.73) in the number of microorganisms. Furthermore, tissue treated with compositions comprising the PHI salts and PHMB showed lower (i.e., practically undetectable) accumulation of IL-8, compared to tissue treated with PHI salts only or PHMB only.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A composition, comprising:
a pharmaceutically acceptable carrier;
an effective amount of an active ingredient of inorganic solids comprising a potassium salt, a zinc salt, a calcium salt, and a rubidium salt; and
an antimicrobial biguanide compound comprising an amount effective to reduce the number of viable microorganisms at a wound site;
wherein each of the salts comprises a pharmaceutically-acceptable anion.

2. The composition of claim 1, wherein the active ingredient of inorganic solids comprises 10-80 parts of potassium ions, 0.00001-20 parts of zinc ions, 0.01-10 parts of calcium ions and rubidium ions in an amount up to 40 parts, said parts being expressed as parts by weight of inorganic solids.

3. The composition of claim 1, wherein the biguanide compound is selected from the group consisting of polyhexamethylene biguanide, chlorhexidine, octenidine, derivatives thereof, and combinations thereof.

4. The composition of claim 1, wherein the composition comprises about 0.1 weight percent to about 0.5 weight percent of the biguanide compound.

5. The composition of claim 1 wherein, relative to a composition without the biguanide component, the composition with the biguanide compound suppresses the accumulation of a polypeptide associated with inflammation.

6. The composition of claim 1 wherein, relative to a composition without the active ingredient, the composition with the active ingredient suppresses the accumulation of a polypeptide associated with inflammation.

7. The composition of claim 5, wherein the polypeptide associated with inflammation comprises a cytokine

8. The composition of claim 7, wherein the cytokine comprises interleukin-8.

9. A wound dressing, comprising:
the composition of claim 1; and
a support.

10. The wound dressing of claim 9, wherein the support comprises a fibrous material, a film, a gel, a foam, a hydrocolloid, an alginate, a hydrogel a polysaccharide paste, a plurality of granules, a plurality of beads or a combination of any two or more of the foregoing.

11. The wound dressing of claim 9, further comprising a backing layer.

12. A method of treating a wound, comprising contacting a wound with the composition of claim 1.

13. A method of treating a wound, comprising contacting a wound with the wound dressing of claim 9.
